(19) United States
(12) Patent Application Publication
(10) Pub. No.: US 2023/0310356 A1
(43) Pub. Date: Oct. 5, 2023

(54) COMPOSITION FOR PREVENTING, AMELIORATING, OR TREATING BRAIN DAMAGE AND MILD COGNITIVE IMPAIRMENT COMPRISING GLUTAMINE AS EFFECTIVE COMPONENT

(71) Applicant: INDUSTRY-ACADEMIC COOPERATION FOUNDATION GYEONGSANG NATIONAL UNIVERSITY, Gyeongsangnam-do (KR)

(72) Inventors: Hyun Joon KIM, Gyeongsangnam-do (KR); Ji Hyung BAEK, Gyeongsangnam-do (KR)

(21) Appl. No.: 18/024,679

(22) PCT Filed: Mar. 25, 2021
(86) PCT No.: PCT/KR2021/003691

§ 371 Date: Mar. 3, 2023

(30) Foreign Application Priority Data
Oct. 8, 2020 (KR) 10-2020-0130170

(51) Int. Cl. A61K 31/198; A61P 25/28

(52) U.S. Cl. CPC A61K 31/198; A61P 25/28

(57) ABSTRACT

A method for ameliorating or treating brain damage and mild cognitive impairment includes administering a composition including glutamine or a salt thereof as effective component to a subject in need thereof. Glutamine as the effective component not only can restore the memory and cognitive ability impaired by stress but also can suppress a damage on nerve cells, reduce the amount of ROS/RNS and other related proteins in brain tissues, and enhance synaptic plasticity. When the animal model of Alzheimer's disease is fed with glutamine, cognitive ability of the animal is improved and accumulation of Aβ42 is suppressed.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2021/003691, filed Mar. 25, 2021, which claims priority to the benefit of Korean Patent Application No. 10-2020-0130170 filed in the Korean Intellectual Property Office on Oct. 8, 2020, the entire contents of which are incorporated herein by reference.

### BACKGROUND

### 1. Technical Field

The present invention relates to a composition for preventing, ameliorating, or treating brain damage and mild cognitive impairment including glutamine as effective component.

### 2. Background Art

Mild cognitive impairment is a state of having cognitive ability loss including memory loss that happens gradually with time but does not affect the individual's ability to carry out everyday activities. As an intermediate stage between normal cognitive aging and dementia, it has a high risk of causing Alzheimer's disease. Being outside the range of objective cognitive decline which naturally occurs according to aging process, mild cognitive impairment corresponds to a clinical pre-stage of major neurocognitive disorder including dementia. Although a decline in memory function appears to be the most serious problem, no emotional response or slow mood change to specific stimulation also suggests the decline in cognitive ability resulting from mild cognitive impairment. However, unlike many major neurocognitive disorders, it is a stage with changes that are good enough to allow relatively normal daily life and activities.

Mild cognitive impairment is characterized by having inconveniences resulting from the decline in cognitive ability, which is mainly caused by memory loss, and there is objective evidence supporting it. Still, a damage on the usual daily activities remains at relatively minor level. Even when a person is found to have mild cognitive impairment under the above diagnostic criteria, it is necessary to figure out to which subtype of mild cognitive impairment the impairment belongs.

As a primary tool for diagnosing mild cognitive impairment, there is a neuropsychological test. However, for determination of more specific subtype, an image test using magnetic resonance imaging (brain MRI) or positron emission tomography (PET) is necessary. Once a person is diagnosed with the disorder as a result of the test, he or she will receive drug therapy using choline esterase inhibitor, an anti-oxidizing agent, an NMDA receptor antagonist, or the like. The likelihood of progression from mild cognitive impairment to Alzheimer's disease or dementia is approximately 10 to 15%.

Meanwhile, being present in large amount in whole body of a human, glutamine is involved in many metabolic processes. It is synthesized from glutamic acid and ammonia, and it is a main transporter of nitrogen in human body and serves as an important energy source in various cells. It is known that, according to oral administration of glutamine, GABA level can be increased in striatal tissues of a brain (FASEB J. 21, 1227-1232 (2007)). In Korean Patent Application Publication No. 2011-0117591, a composition for preventing or treating depression including glutamine is described. However, so far there is no disclosure of a composition for preventing, ameliorating, or treating brain damage and mild cognitive impairment including glutamine as effective component as it is described in the present invention.

### SUMMARY

The present invention is devised under the circumstances that are described in the above, and provided in the present invention is a composition for preventing, ameliorating, or treating brain damage and mild cognitive impairment including glutamine as effective component. According to ORT (Objective Recognition Test) and OLT (Objective Location Test), it was found that the stress group (STR) induced to have brain damage and mild cognitive impairment by applying stress has lower object memory and lower location memory compared to the normal group, but the group with glutamine diet (STR+Gln), which is the effective component of the present invention, maintains the cognitive ability that is comparable to the ability of the normal group. STR+Gln group shows no damage in neurons while STR group has a damage in neurons. STR+Gln group also shows lower content of ROS/RNS and other related proteins in brain tissues and synaptic plasticity is enhanced in this group. As a result of the glutamine diet, the animal model of Alzheimer's disease (3× TG) exhibited improved cognitive ability and suppressed accumulation of Aβ42. Accordingly, the present invention is completed.

To achieve the object described in the above, the present invention provides a functional health food composition for preventing or ameliorating brain damage and mild cognitive impairment including, as effective component, glutamine or a salt thereof that is acceptable for use in food product.

The present invention further provides a pharmaceutical composition for preventing or treating brain damage and mild cognitive impairment including, as effective component, glutamine or a pharmaceutically acceptable salt thereof.

The present invention further provides a quasi-drug for preventing or ameliorating brain damage and mild cognitive impairment including, as effective component, glutamine or a pharmaceutically acceptable salt thereof.

The present invention relates to a composition for preventing, ameliorating, or treating brain damage and mild cognitive impairment including glutamine as effective component. It was found that the control group applied with stress shows a decline in memory and cognitive ability but the test group with glutamine supplementation diet shows cognitive ability that is restored almost to the level of normal group, has suppressed damage on nerve cells, reduced content of ROS/RNS and other related proteins in brain tissues, and enhanced synaptic plasticity. It was also found that, as a result of the glutamine diet, the animal model of Alzheimer's disease exhibits improved cognitive ability and suppressed accumulation of A→2. Accordingly, it is recognized that glutamine as the effective component of the present invention is effective for chronic brain damage and mild cognitive impairment.

### BRIEF DESCRIPTION OF THE DRAWINGS

FIGS. 1A to 1D show the result of determining (B) a change in bodyweight, (C) a change in food intake, and (D) a change in plasma corticosterone level, all after the application of chronic immobilization stress (CIS) and glutamine supplementation diet. FIG. 1A is a diagram showing the experimental schedule. *, **, and *** in FIG. 1B indicate that there is a statistically significant decrease in the bodyweight of STR compared to CTL, in which * has p<0.05, ** has p<0.01, and *** has p<0.001. * and *** in FIG. 1D indicate that there is a statistically significant difference in the corticosterone level of CTL, CTL+Gln, or STR+Gln compared to STR, in which * has p<0.05 and *** has p<0.001.

FIGS. 2A and 2B show the result of behavior analysis including ORT (object recognition test) and OLT (object location memory test) after the application of chronic immobilization stress (CIS) and glutamine supplementation diet. * and ** in indicate that, compared to STR, there is a statistically significant difference in the discrimination index (DI) in ORT or OLT which has been carried out as behavior test for CTL, CTL+Gln, or STR+Gln, in which * has p<0.05 and ** has p<0.01.

FIGS. 3A and 3B show the result of determining a morphological change in hippocampus after the application of chronic immobilization stress (CIS) and glutamine supplementation diet. Specifically, FIG. 3A is a photographic image obtained by Nissl staining of brain tissue specimen to determine the width of stratum pyramidale CA1 in hippocampus, in which the bar length is 100 μm and the arrow represents small and dark-stained cells. FIG. 3B shows the result of measuring the width value of stratum pyramidale CA1 in hippocampus, in which *** indicates that, compared to STR, there is a statistically significant difference in the width of stratum pyramidale CA1 in hippocampus of CTL, CTL+Gln, or STR+Gln, in which *** has p<0.001.

FIGS. 4A to 4C show the result of determining (A) a change in ROS/RNS in plasma, (B) a change in ROS/RNS in the PFC (prefrontal cortex), and (C) a change in ROS/RNS in the hippocampus, all after the application of chronic immobilization stress (CIS) and glutamine supplementation diet. *, **, and *** indicate that there is a statistically significant difference in the ROS/RNS of CTL, CTL+Gln or STR+Gln compared to STR, in which * has p<0.05, ** has p<0.01, and *** has p<0.001.

FIGS. 5A to 5E show the result of determining a change in content of the proteins that are related to reactive oxygen/nitrogen species in microglia present in the infralimbic cortex and a change in synaptic puncta after the application of chronic immobilization stress (CIS) and glutamine supplementation diet. In FIGS. 5A to 5D, cell count including iNOS, nNOS, p47phox, and p67phox positive cells is shown, in which the arrowhead ( [symbol] ) represents double-positive cells which react with two types of antibodies and the arrow (→) represents single-positive cells. FIG. 5E shows the determination result of synaptic puncta from the cells (∘) co-localized in PSD-95 and synaptophysin.

FIGS. 6A to 6F show the result of determining a change in content of the proteins that are related to reactive oxygen/nitrogen species in the hippocampal CA1 region after the application of chronic immobilization stress (CIS) and glutamine supplementation diet. In FIGS. 6A to 6E, cell count including iNOS, nNOS, p47phox, and p67phox cells is shown, in which Iba1 represents a microglia cell marker and NeuN represents a neuron marker. FIGS. 6A to 6D are the image obtained by an analysis of stratum radiatum, and the FIG. 6E is the image obtained by an analysis of stratum pyramidale. The arrowhead ( [symbol] ) represents double-positive cells which react with two types of antibodies and the arrow (→) represents single-positive cells. FIG. 6F shows the determination result of synaptic puncta from the cells (∘) co-localized in PSD-95 and synaptophy sin.

FIGS. 7A and 7B show (A) the schedule for testing the effect of glutamine to prevent cognitive impairment and (B) a diagram of the object recognition test (ORT), in which an animal model of Alzheimer's disease (3×TG) has been employed for the test.

FIGS. 8A and 8B show the result of determining a change in object recognition ability depending on different age (i.e., months) of the mouse, i.e., C57BL/6 (NonTg) and 3×TG-AD (3×TG), and various diets. In FIG. 8A, DI (discrimination index) value for various diets is given, in which NonTg represents the normal group, NonTg+Gln represents the normal group with glutamine diet, 3×TG represents the group with induced Alzheimer's disease, and 3×TG+Gln represents the group with induced Alzheimer's disease but treated with glutamine diet. In FIG. 8A,* indicates that, compared to the result from the 2-months old animal, there is a statistically significant difference according to One-way ANOVA (Tukey post-hoc test), in which * has p<0.05. In FIG. 8B, a difference in recognition ability, which has been measured by ORT, between the 2-months old animal and the 6-months old animal is shown. * indicates that there is a statistically significant difference according to Student's t-test, in which * has p<0.05.

FIG. 9 shows the result of determining a change in Aβ42, i.e., a neuronally derived exosome, in blood after application of glutamine diet. * indicates that, according to Student's t-test, content of Aβ42 as a neuronally derived exosome has a statistically significant increase in the blood of 3×TG group (n=3) compared to NonTg group (n=5), in which * has p<0.05. #indicates that, according to Student's t-test, content of β42 as an a neuronally derived exosome has a statistically significant decrease in the blood of 3×TG+Gln group (n=3) compared to 3×TG, in which #has p<0.05.

FIGS. 10A and 10B show (A) a diagram of the Y-maze that has been used in the present invention and (B) a diagram explaining the method of carrying out ORT-OLT.

FIGS. 11A to 11D show the result of determining the effect of improving cognitive ability as a result of oral administration of the glutamine of the present invention. Specifically, FIG. 11A shows the schedule for determining the effect of improving cognitive ability, FIG. 11B shows the result of Y-maze analysis, FIG. 11C shows the result of ORT analysis, and FIG. 11D shows the result of OLT analysis.

FIGS. 12A to 12C show the result of determining the effect of improving cognitive ability as a result of oral administration of the glutamine of the present invention, in which the oral administration was made after applying chronic immobilization stress. Specifically, FIG. 12A shows the schedule for determining the effect of improving cognitive ability as a result of oral administration of the glutamine of the present invention, in which the oral administration was made after applying chronic immobilization stress. FIG. 12B shows the result of ORT analysis and FIG. 12C shows the result of OLT analysis.

### DETAILED DESCRIPTION

The present invention relates to a functional health food composition for preventing or ameliorating brain damage and mild cognitive impairment including, as effective component, glutamine or a salt thereof that is acceptable for use in food product.

It is characterized in that the brain damage and mild cognitive impairment are caused by either stress or accumulation of amyloid beta 42 (Aβ42).

The mild cognitive impairment is preferably a decline in one or more function selected from memory, cognitive ability, and learning ability, but it is not limited thereto.

The functional health food composition of the present invention may be produced as any one selected from a pill, a tablet, a capsule, a powder preparation, powder, a granule, a candy, a syrup, and a drink, or the production may be carried out by adding it as an ingredient of a food product. The functional health food composition can be suitably produced according to a general method. As an example of the food product to which the effective component of the present invention can be added, it can be in the form that is any one selected from meat, sausage, bread, chocolate, candies, snacks, biscuits, pizza, ramen, other noodles, gums, dairy products including ice cream, various kinds of soup, beverage, tea, drink, alcohol beverage, and vitamin complex, and all functional health food products in general sense are included therein. The functional health food composition of the present invention may further include various nutritional supplements, a vitamin, a mineral (i.e., electrolyte), a synthetic or natural flavor, a coloring agent, an enhancing agent (i.e., cheese, chocolate, or the like), pectinic acid and a salt thereof, alginic acid and a salt thereof, an organic acid, a protective colloidal thickening agent, a pH adjusting agent, a stabilizer, a preservative, glycerin, alcohol, and a carbonating agent used for carbonated drink. Other than those, natural fruit juice or fruit pulp for producing vegetable drink may be additionally included. Those ingredients may be used either singly or in combination thereof. The functional health food composition of the present invention may further include various flavoring agents, natural carbohydrates, or the like as an additional component. Examples of the natural carbohydrates include monosaccharides like glucose and fructose, disaccharides like maltose and sucrose, polysaccharides like dextrin and cyclodextrin, and sugar alcohols like xylitol, sorbitol, and erythritol. As a sweetening agent, natural sweetening agent like thaumatin and stevia extract and synthetic sweetening agent like saccharine and aspartame can be used.

The present invention further relates to a pharmaceutical composition for preventing or treating brain damage and mild cognitive impairment including, as effective component, glutamine or a pharmaceutically acceptable salt thereof.

In addition to the effective component described above, a pharmaceutically acceptable carrier, vehicle, or diluent may be additionally included. The pharmaceutical composition of the present invention can be administered either orally or parenterally. In case of parenteral administration, it is preferable to choose external application on skin, or intraperitoneal, rectal, intravenous, muscular, or subcutaneous injection, but it is not limited thereto.

The pharmaceutical composition of the present invention may be produced by using a diluent or a vehicle like a filling agent, a bulking agent, a binding agent, a wetting agent, a disintegrating agent, and a surfactant. Examples of the solid preparation for oral administration include a tablet, a pill, a powder preparation, a granule, and a capsule. The solid preparation is produced by mixing at least one compound with one or more vehicles such as starch, calcium carbonate, sucrose, lactose, or gelatin. Furthermore, other than simple vehicles, a lubricating agent such as magnesium stearate or talc is also used. As for the liquid preparation for oral administration, a suspension, a solution preparation for internal use, an emulsion, a syrup preparation, or the like can be mentioned. Other than water or liquid paraffin commonly used as a simple diluent, various kinds of a vehicle such as moisturizing agent, sweetening agent, aromatic agent, or preservatives may be included. Examples of a preparation for parenteral administration include a sterilized aqueous solution, a non-aqueous preparation, a suspension preparation, an emulsion preparation, a freeze-dried preparation, and a suppository preparation. As a non-aqueous preparation or a suspending preparation, propylene glycol, polyethylene glycol, or vegetable oil such as olive oil, and injectable ester such as ethylolate can be used. As a base for a suppository preparation, witepsol, macrogol, tween 61, cacao fat, laurin fat, glycerol, gelatin, or the like can be used.

The composition according to the present invention is administered in a pharmaceutically effective amount. As described herein, the expression “pharmaceutically effective amount” means an amount sufficient for treating a disorder at reasonable benefit-risk ratio that can be applied for a medical treatment. The effective dose level may be determined based on a type or severeness of a disorder of a patient, activity of a pharmaceutical, sensitivity to a pharmaceutical, administration period, administration route, excretion ratio, time period for therapy, elements including a pharmaceutical used in combination, and other elements that are well known in the medical field. The composition of the present invention can be administered as a separate therapeutic agent, or it can be used in combination with other therapeutic agent. It can be administered in order or simultaneously with a conventional therapeutic agent. It can be also administered as single-dose or multi-dose. It is important to administer an amount which allows obtainment of the maximum effect with minimum dose while considering all of the aforementioned elements without having any side effect, and the dosage can be easily determined by a person skilled in the pertinent art.

The dosage of the composition of the present invention may vary in a broad range depending on bodyweight, age, sex, health state, diet of a patient, administration period, administration method, excretion rate, and severeness of disorder.

The present invention still further relates to a quasi-drug for preventing or ameliorating brain damage and mild cognitive impairment including, as effective component, glutamine or a pharmaceutically acceptable salt thereof.

When glutamine or a pharmaceutically acceptable salt thereof as the effective component of the present invention is used as an additive for quasi-drug, the effective component may be directly added by itself or used in combination of other quasi-drug or components of quasi-drug, and it may be suitably used according to a common method. Blending amount of the effective component may be suitably determined based on the purpose of use.

Hereinbelow, the present invention is explained in greater detail in view of the Examples. However, the following Examples are given only for more specific explanation of the present invention and it would be evident to a person who has common knowledge in the pertinent art that the scope of the present invention is not limited by them.

### EXAMPLES

### Example 1. Determination of Change in Bodyweight, Food Intake, and Corticosterone Content after Application of Chronic Immobilization Stress (CIS) and Glutamine Supplementation

### [Animal Model]

In the present invention, C57BL/6 male mice (7-weeks old, KOATECH, South Korea) were kept under standard conditions (i.e., temperature of 22 to 24° C., humidity of 50 to 70%, and 12-hour light/dark cycle with lighting on 6 AM), and the animals were allowed to have free access to diets and water. The animals used for the present invention were handled according to the protocol (GNU-161128-M0068) acknowledged by Gyeongsang National University Institutional Animal Care and Use Committee (GNU IACUC), which follows the guidelines of National Institutes of Health (NIH, Bethesda, MD, USA).

Control (CTL) is a group which received normal diet (common diet with nutritional balance) without any stress, CTL+Gln group is a normal diet group supplemented with glutamine (150 mg/kg of feed) and without any stress, STR is a normal diet group under stress, and STR+Gln group is a normal diet group supplemented with glutamine (150 mg/kg of feed) under stress. The stress group (STR) was individually brought by force to a restrainer for 2 hours every day (i.e., between 2 PM and 4 PM) so that the animals are applied with chronic immobilization stress for 15 days.

The animal test carried out in the present invention includes 3 cohorts, i.e., the first cohort consists of 28 animals in total, i.e., 7 animals per group, the second cohort consists of 51 animals in total, i.e., 12 to 13 animals per group, and the third cohort consists of 32 animals in total, i.e., 8 animals per group.

By using the above first cohort, a change in bodyweight and a food intake were measured for each animal model group (28 animals in total, i.e., 7 animals per group), in which the measurement was made, with an interval of 2 days, for 15 days of applying stress.

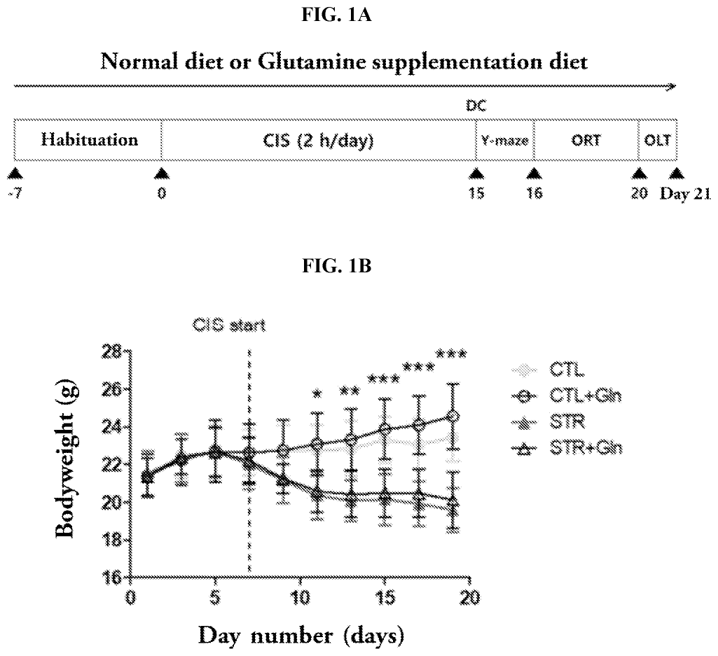

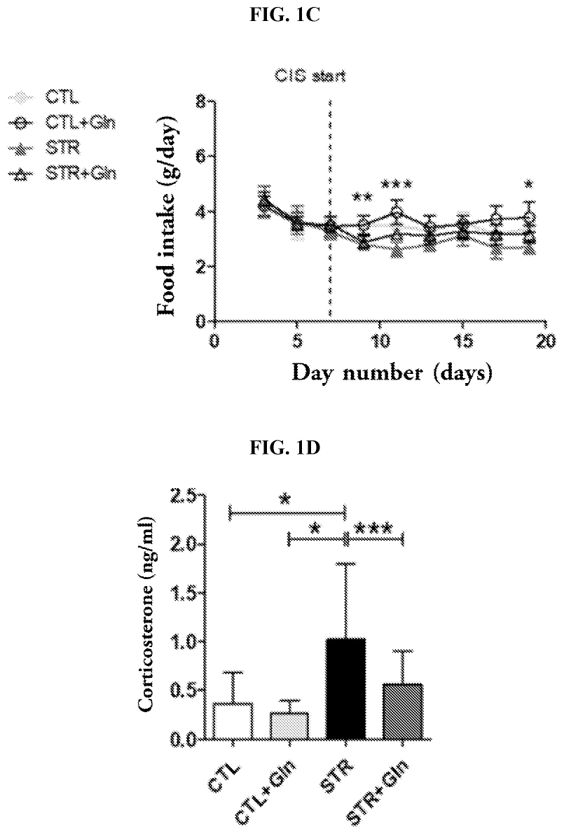

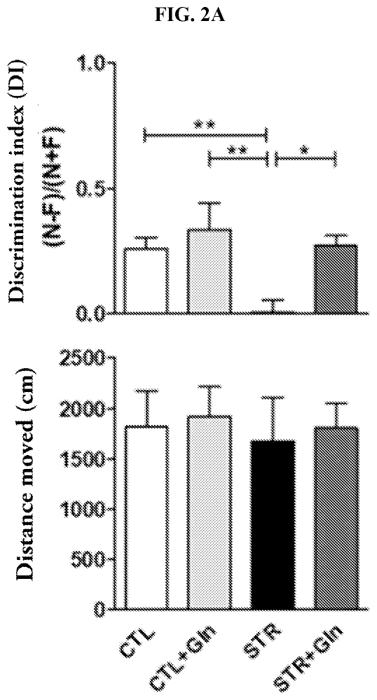

As the result is illustrated in FIGS. 1A to 1D, it was found that the bodyweight of CTL and CTL+Gln, which had not been applied with any stress, tends to gradually increase with time, but the bodyweight of STR and STR+Gln, which had been applied with stress, tends to decrease with time. In this regard, the change in bodyweight was hardly affected by glutamine supplementation, indicating that there is no difference between the presence and absence of glutamine supplementation. In terms of the food intake, however, there was almost no difference so that CTL, CTL+Gln, STR and STR+Gln groups consumed the feed at similar level.

Meanwhile, as a result of determining a change in plasma corticosterone level, it was found that higher corticosterone level is obtained from the stress group (STR) compared to Control (CTL) as it is illustrated in FIG. 1D. On the other hand, it was also found that there is a statistically significant decrease in corticosterone content in the glutamine supplementation group of the present invention (STR+Gln).

### Example 2. Behavior Analysis by ORT (Object Recognition Test) and OLT (Object Location Memory Test) after Application of Chronic Immobilization Stress (CIS) and Glutamine Supplementation

By using the cohorts of Example 1, behavior analysis was carried out. After applying the stress for 15 days, behavior analysis was immediately carried out.

ORT includes three separate sessions, i.e., habituation, familiarization, and test.

During 2 days of the habituation, the animals were allowed to stay in an empty open space for 10 minutes a day. After the habituation, they were subjected to familiarization by placing two identical objects in the open space. Mice of each test group were allowed to explore freely the object for 10 minutes. On the next day, one of the two objects was replaced with a novel object, and the test session was repeated. For each session, initial 5 minutes were taken for the analysis.

Discrimination index (DI) in ORT was then calculated according to the following formula (1).

DI(Discrimination Index) in ORT=[(Time spent for exploring novel object(N))−Time spent for exploring familiar object(F)]/[Total object exploration time(N+F)] Formula (1)

When DI is 0, it means that the preference is the same for both objects. On the other hand, when DI is negative (−), it means that the novel object is disliked by a testee, and greater negative value indicates higher level of dislike.

After the ORT, OLT was carried out in the same open space as described in the above. After translocating the novel object of above ORT to another place, the test was carried out in the same manner as the ORT. Discrimination index (DI) in OLT was then calculated according to the following formula (2).

DI(Discrimination Index) in OLT=[(Time spent for exploring translocated object(T))−Time spent for exploring familiar object(F)]/[Total object exploration time(T+F)] Formula (2)

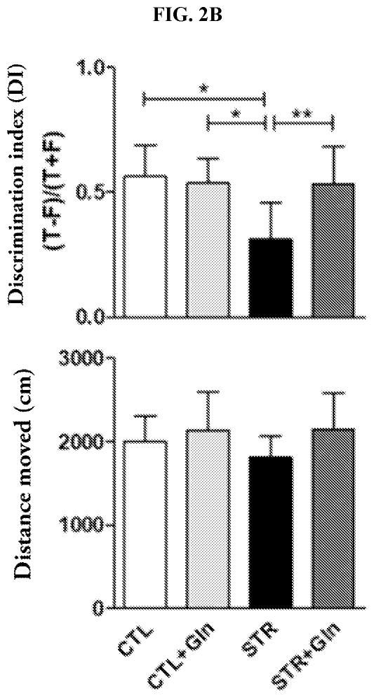

As the result is illustrated in FIGS. 2A and 2B, it was found that the stress group (STR) has a statistically significant decrease in the discrimination index (DI) in ORT or OLT when compared to CTL or CTL+Gln. On the other hand, a higher DI was obtained from the glutamine supplementation group of the present invention. Meanwhile, as there was almost no difference in the travel distance in ORT and OLT among different test groups, it was recognized that the aforementioned change is caused not by a decrease in mobility but by a difference in the cognitive ability instead.

### Example 3. Determination of Morphological Change in Hippocampus after Application of Chronic Immobilization Stress and Glutamine Supplementation

Any morphological change in hippocampus caused by the application of chronic immobilization stress and glutamine supplementation was determined by brain tissue staining of hippocampal region.

Specifically, a section (i.e., brain tissue specimen) obtained after mounting for 5 minutes in 0.1% cresyl violet which has been dissolved in 0.2% acetic acid followed by air-drying was stained. Stained section was washed, subsequently immersed for 30 seconds in 80% (v/v) ethanol, again immersed for 30 seconds in 90% (v/v) ethanol, and again immersed twice for 30 seconds in 95% (v/v) ethanol. It was again immersed twice for 30 seconds in 100% (v/v) ethanol for dehydration. Thereafter, clearing was carried out by treating the section with xylene, twice for 1 minute, applied with xylene mounting solution (xylene:Permount=1:1), and dried in a hood.

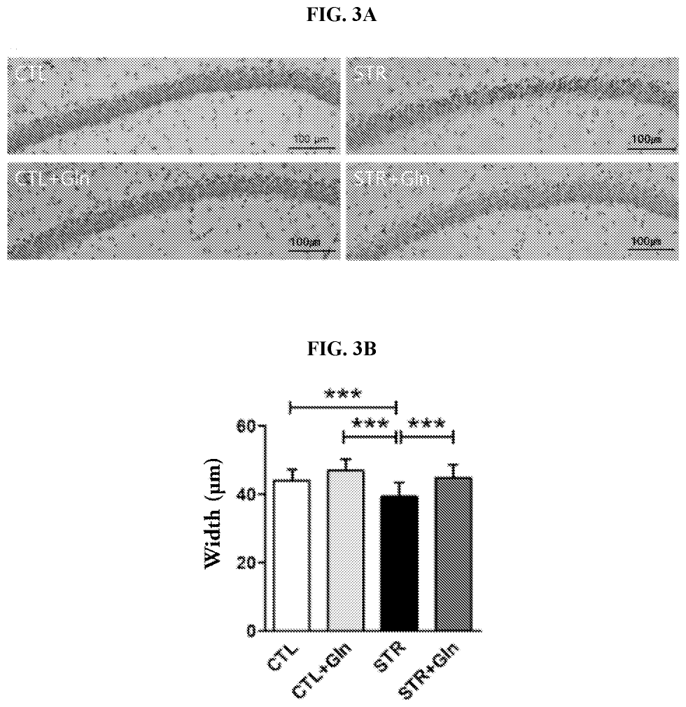

As the result is illustrated in FIGS. 3A and 3B, it was found that the width of CA1-SP (stratum pyramidale CA1) is reduced by the application of stress, while the width of CA1-SP is restored by the supplementation of glutamine of the present invention. Based on this result, it was confirmed that the brain damage caused by application of stress can be ameliorated/treated by supplementation of glutamine.

### Example 4. Determination of Change in ROS/RNS Levels in Plasma, PFC, and Hippocampus after Application of Chronic Immobilization Stress and Glutamine Supplementation

Fifty-one model animals of the second cohort, i.e., 12 to 13 animals per group, were not subjected to any behavior analysis but immediately sacrificed after completion of the last immobilization stress. Then, ROS/RNS levels in the plasma, PFC, and hippocampus were examined. Specifically, ROS/RNS was examined by using ROS/RNS assay kit (STA-347; Cell Biolabs, San Diego, CA, USA) and following the manufacturer's protocol.

PFC and hippocampal tissues were subjected to tissue homogenization by using Bullert blender after adding glass beads to RIPA (radioimmunoprecipitation assay) buffer solution. After the ultrasonication for 2 minutes, they were centrifuged for 10 minutes at conditions including 4° C. and 12,000×g. Then, the measurement of fluorescence intensity using Tecan Infinite F200 PRO MicroReader was repeated 3 times.

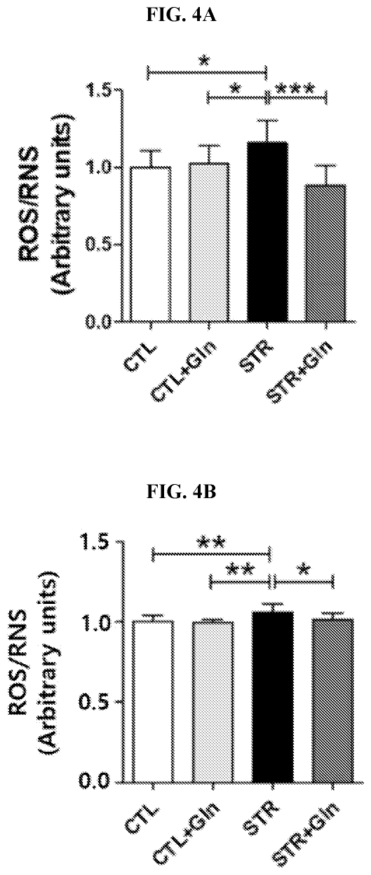

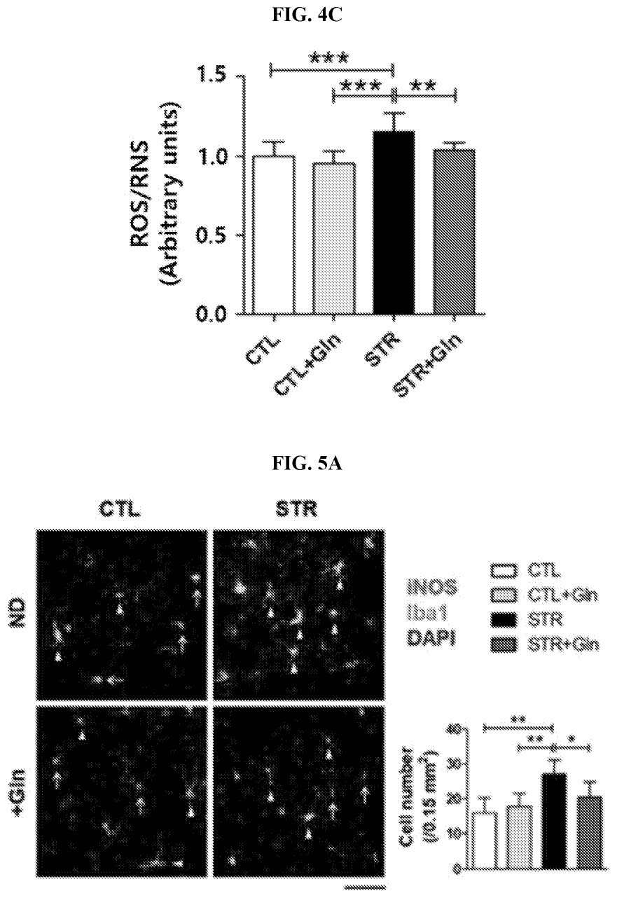

As the result is illustrated in FIGS. 4A to 4C, it was found that, in all of the plasma, PFC, and hippocampal tissues, the higher level of active oxygen/nitrogen species is obtained after the application of stress. On the other hand, lower level of active oxygen/nitrogen species is shown from the group which has been supplemented with glutamine of the present invention.

### Example 5. Determination of Change in Protein Expression Level after Application of Chronic Immobilization Stress and Glutamine Supplementation

To determine the specific changes in the brain tissues after glutamine supplementation, double staining was carried out with a cell marker so that the proteins related to a change in ROS/RNS content (i.e., iNOS; nNOS; and p47phox and p67phox as a subunit of NOX) with cell markers are analyzed by double-IHC (immunohistochemistry).

Twenty-four hours after applying the last stress, the mouse was anesthetized with avertin, and perfused with PBS (pH 7.4) and 4% formaldehyde. After that, the brain was removed, fixed, and cut to a thickness with 40 μm. It was then incubated overnight after the addition of each antibody, nNOS (sc-5302, 200 μg/ml, Santa Cruz, Dallas, TX, USA, 1:50), iNOS (sc-7271, 200 μg/ml, Santa Cruz, 1:20), p47phox (sc-17845, 200 μg/ml, Santa Cruz, 1:20), p67phox (15551-1-AP, 43 μg/150 μl, Proteintech, Rosemont, IL, USA, 1:100), PSD-95 (postsynaptic density-95) (ab12093, 1 mg/ml, Abcam, Cambridge, UK, 1: 200), and synaptophysin (ab14692, 0.65 mg/ml, Abcam, 1: 200). As a cell marker, Iba1 (Ionized calcium-binding adapter molecule 1, ab5076, 0.5 mg/ml, Abcam, 1: 200), NeuN (neuronal nuclei, MAB377, 30 μg/ml, Merck Millipore, St. Louis, MO, USA, 1: 200), and GAD2 (glutamate decarboxylase, #3988, Cell Signaling, Danvers, MA, USA, 1: 100) were used. The brain tissue specimens were incubated with AlexaFluor 594 and 488-conjugated secondary antibody (2 mg/ml, Invitrogen, Carlsbad, CA, USA, 1: 1000). After that, the analysis was carried out using a confocal microscope equipped with Olympus disc spinning unit, and Image J Program.

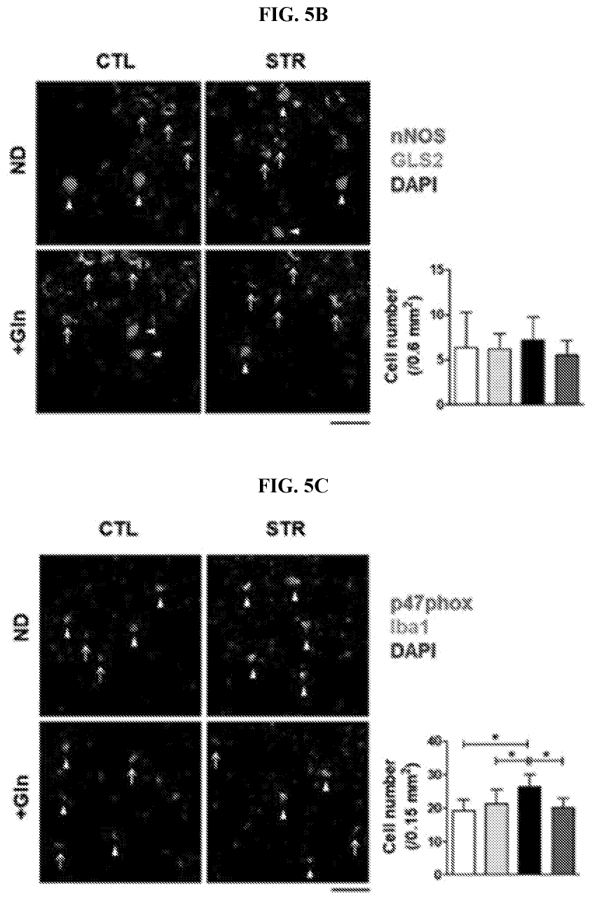

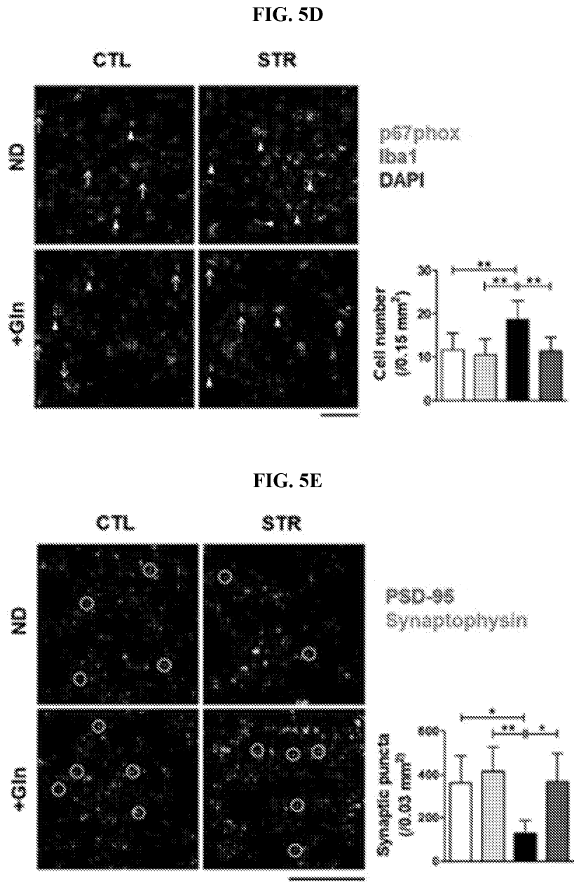

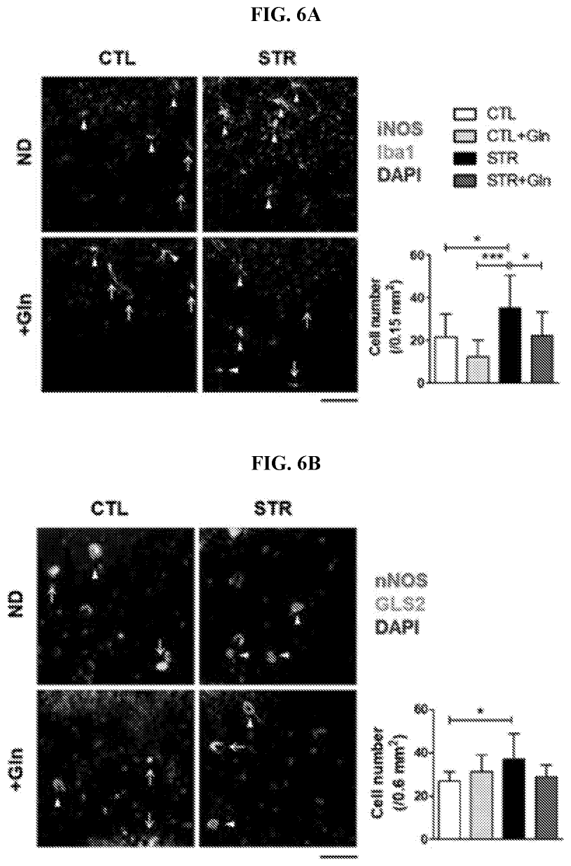

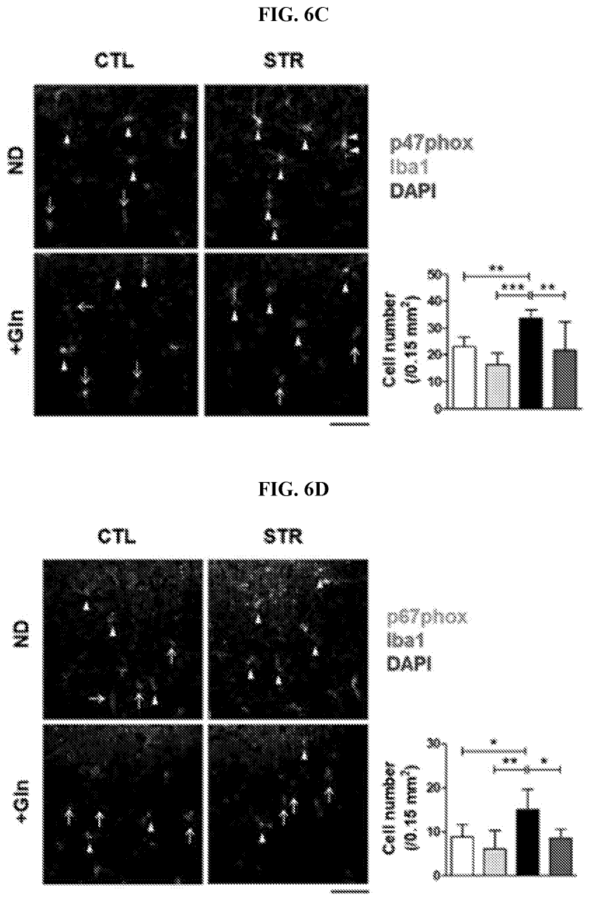

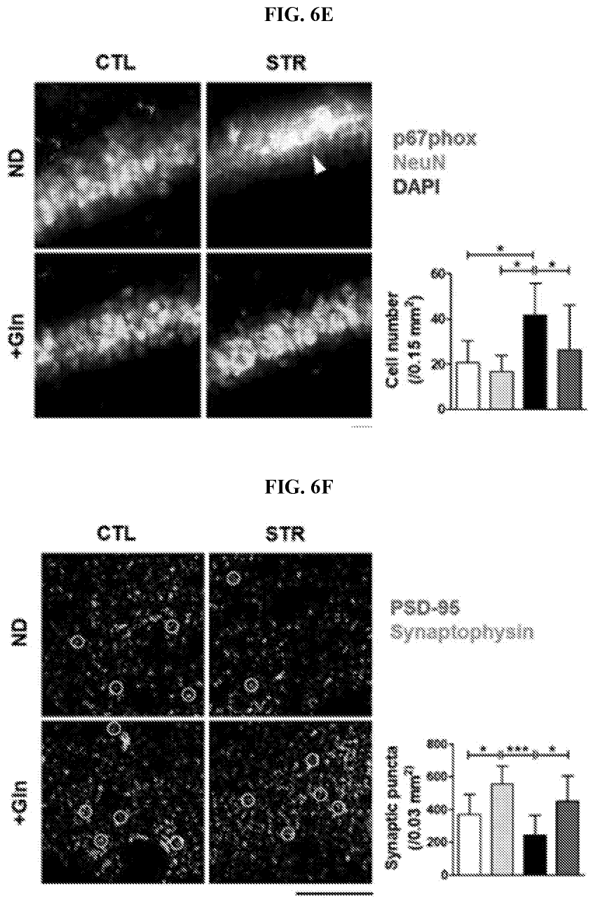

As the result is illustrated in FIGS. 5A to 6F, it was found that the expression amount of iNOS, nNOS, and p47phox and p67phox as a subunit of NOX becomes statistically significant increment in the microglial cells in infralimbic cortex and hippocampal CA1 region of the stress group. On the contrary, a statistically significant decrement was shown in the group with glutamine supplementation diet.

Furthermore, as a result of examining the co-localization of PSD-95 and synaptophysin, which enables the determination of synaptic plasticity, it was found that the synaptic puncta are significantly increased by glutamine (FIGS. 5E and 6F).

### Example 6. Determination of the Effect of Glutamine to Prevent Mild Cognitive Impairment in Model of Alzheimer's Disease

(1) Establishment of Model of Alzheimer's Disease with Glutamine Diet

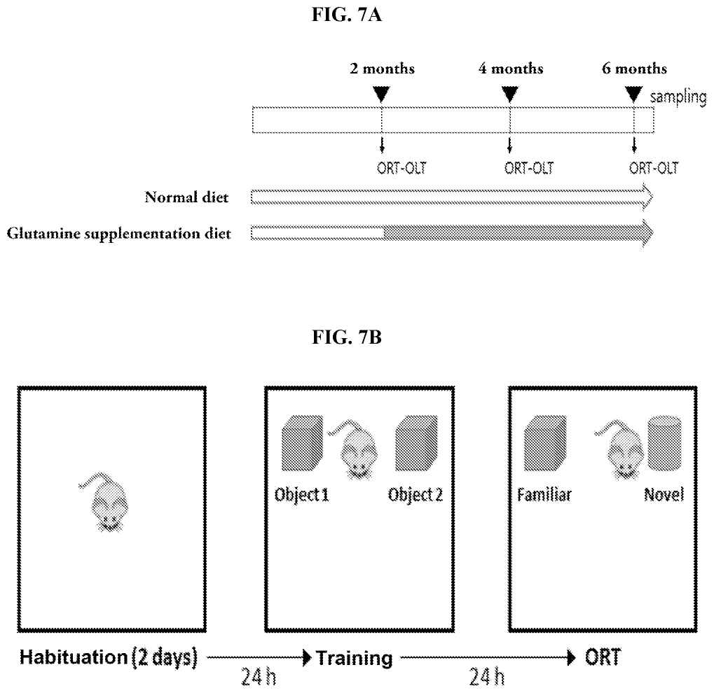

As an animal model for cognitive ability evaluation, a 7-weeks old 3×TG-AD female mouse (8 mice. model of Alzheimer's disease) and a C57BL/6 female mouse (10 mice, control) were selected. The animals of each type were divided into 2 groups, and each group was provided with regular animal feed (i.e., normal diet, ND) or glutamine feed (Gln). The glutamine diet group was allowed to consume the feed containing 150 mg glutamine/kg diet, from 2-months old stage to the end of test (i.e., 6 to 7-months old stage). Health state of the animal was checked by measuring, once a week, the bodyweight of the animal and food intake (FIG. 7A).

### (2) Cognitive Ability Test

Cognitive ability test of the mouse as an animal model established above was determined by ORT (object recognition test). The animal was habituated in a box for 2 days. On Day 3, it was allowed to explore two identical objects for 10 minutes. On Day 4, one of the two objects was replaced with a novel object, and then, the animal was allowed to explore further for 5 minutes (FIG. 7B). The time spent by animal for exploring after approaching within 1 cm from the periphery of both objects was measured. After carrying out the calculation using Formula (3) below, DI (discrimination index) was calculated and compared to each other. In the present invention, a different novel object was employed for carrying out each test.

DI=[(Time spent for exploring novel object(N)−Time spent for exploring familiar object(F))]/[(Time spent for exploring novel object(N)+Time spent for exploring familiar object(F))] Formula (3)

### (3) Determination of Effect of Glutamine Diet on Delayed Onset of Mild Cognitive Impairment in Animal Model of Alzheimer's Disease

Animals were subjected to the cognitive ability test when they are 2-, 4-, or 6-months old. Analysis was made by classifying the animals into the following groups: Normal group (NonTg, n=5), Normal group with glutamine diet (NonTg+Gln, n=5), Group with induced Alzheimer's disease (3×TG, n=4), and Group with induced Alzheimer's disease but under glutamine diet (3×TG+Gln, n=4).

As a result, in NonTg mouse group as a normal group, there was no decrease in cognitive ability until the 6-months old stage, and there was also no change caused by the glutamine diet. Compared to the 2-weeks old, the 6-weel old 3×TG-AD mouse group showed remarkably impaired object recognition ability. On the other hand, no significant impairment in cognitive ability was observed from the glutamine diet group.

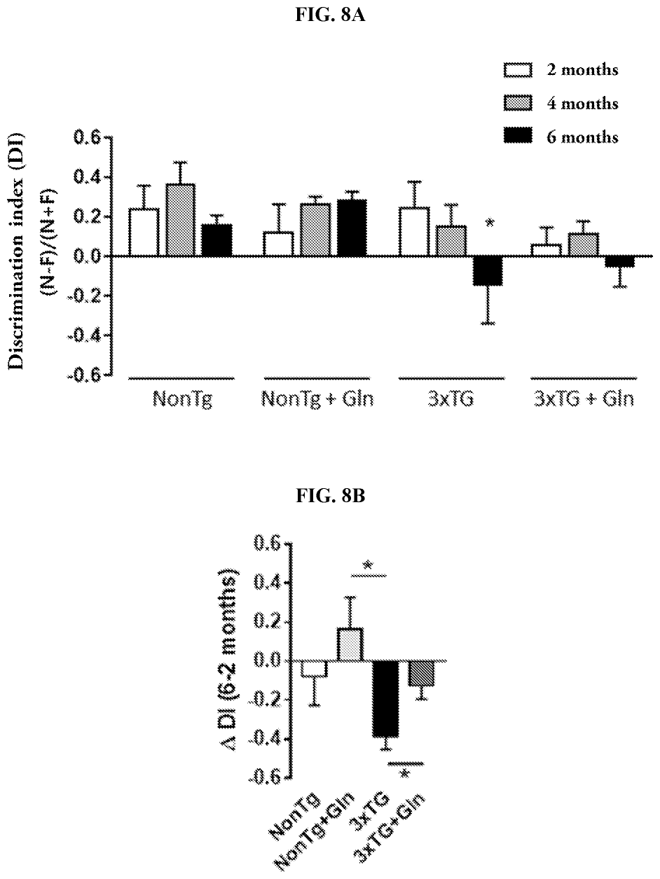

Compared to the 2-weeks old, the 6-weel old 3×TG-AD mouse group showed a lower DI value in ORT, i.e., 0.4 less on average from each individual, representing the largest change among the above test groups. On the other hand, 3×TG-AD group with glutamine diet showed a decrease of only 0.1, representing no significant change compered to NonTg group. Thus, it was confirmed that the glutamine diet in animal model of Alzheimer's disease can delay the onset of mild cognitive impairment (FIGS. 8A and 8B).

### (4) Delayed Accumulation of Amyloid Beta 42 (Aβ42) by Glutamine Diet

After the cognitive ability test with 6-months old animals, the mouse was sacrificed using carbon dioxide and a change in neuronally derived blood exosome (NDE) protein was determined. Total exosomes were separated from serum, and, after a reaction for 1 hour with anti-CD171 antibody, they were reacted for 1 hour with streptavidin-fused magnetic beads. After that, elution was carried out using 0.05 M glycine-HCl (pH 3.0), and then addition of 1 M Tris-HCl (pH 8.0) was made. Analysis was then made by using amyloid beta 42 (Aβ42) mouse ELISA kit.

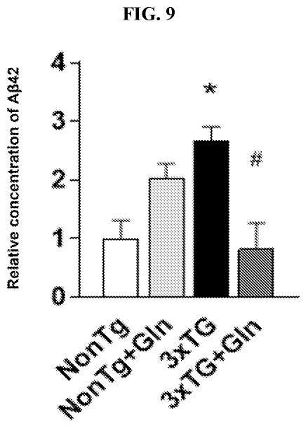

As a result, it was found that an increase in Aβ42 is obtained from NDE of female 3×TG-AD while such increase in Aβ42 is suppressed in the glutamine diet group. It was confirmed based on this result that, as the body accumulation of Aβ42 is suppressed by glutamine diet, the effect of preventing the loss of cognitive ability is exhibited (FIG. 9).

### (5) Determination of the Effect of Glutamine on Improving Cognitive Ability

To determine if there is also an effect of orally administered glutamine on improving the cognitive ability of a normal mouse, 2 hours later glutamine administration, any improvement in cognitive ability was examined. The cognitive ability was tested by using Y-maze for testing short-term memory and ORT and OLT (object location recognition test) for testing long-term memory.

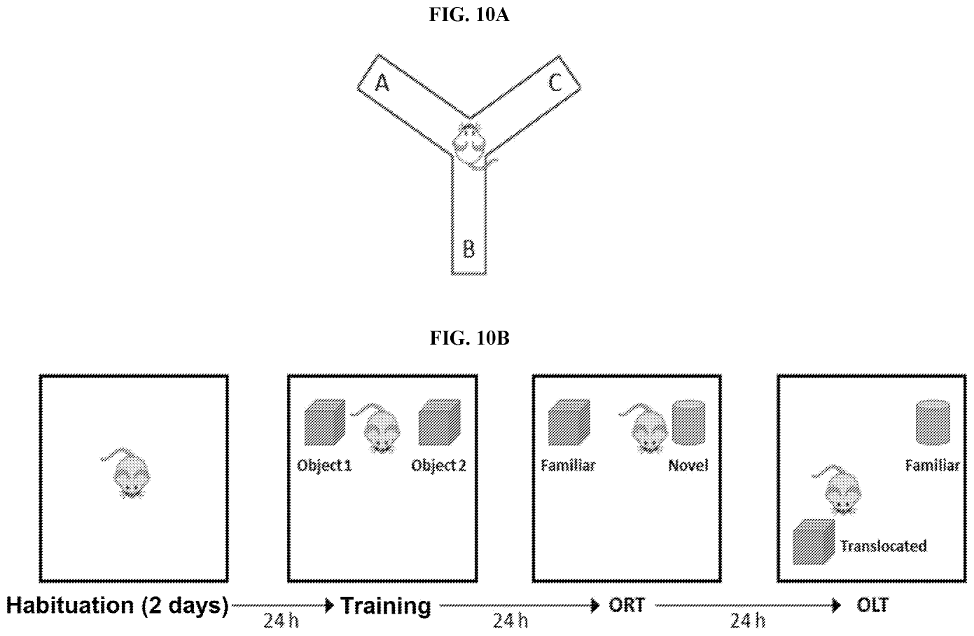

Y-maze: To a Y-shaped maze consisting of 3 arms that are spread at 120° angle from each other and designated as A, B, and C, respectively, a mouse was introduced. Then, the spontaneous alternation indicating an entrance into different arms, 3 times consecutively, was applied to the following Formula (4) and the calculation was made (FIG. 10A).

Three consecutive spontaneous alternation=[(Number of consecutive entry into different arms)/(Total number of arm entries−2)]×100 Formula (4)

ORT/OLT: ORT was carried out for 10 minutes as described above, and, 24 hours later, one of the objects was translocated to a new spot and the animal was allowed to explore again for 5 minutes (FIG. 10B).

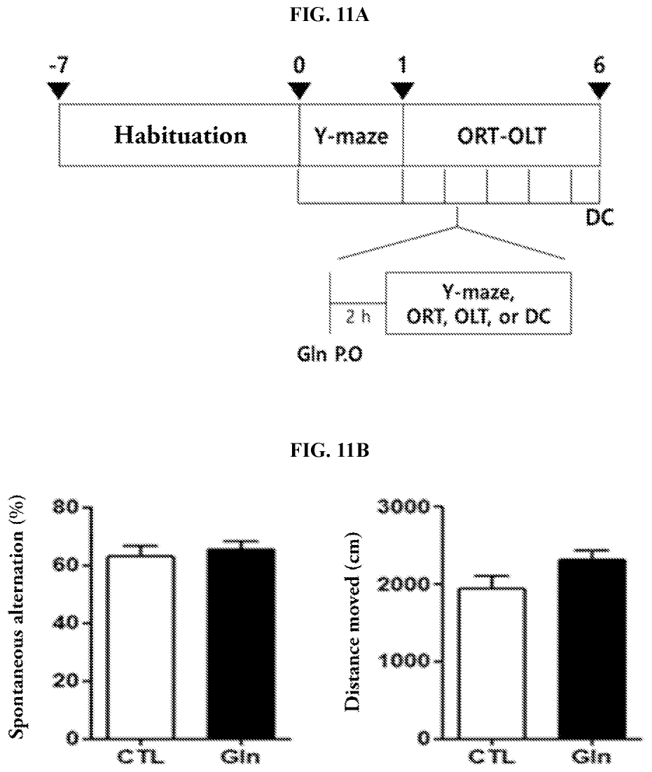

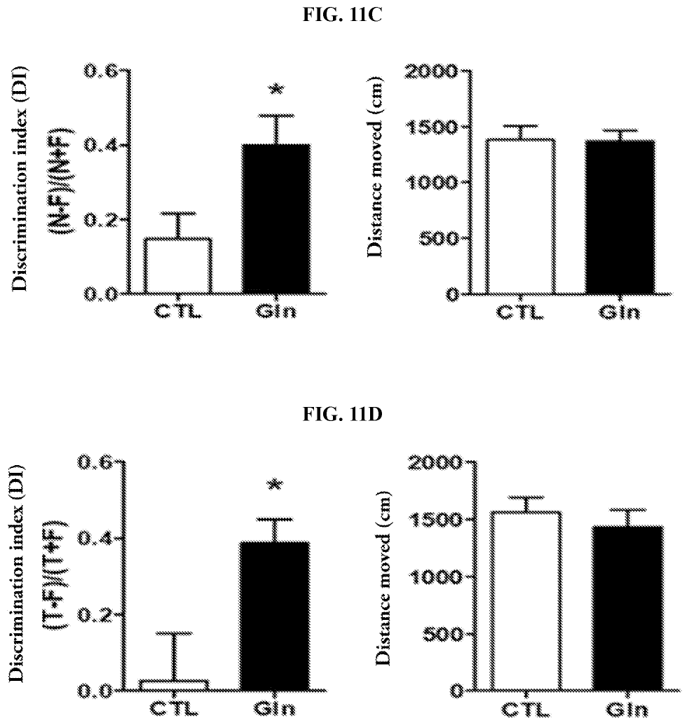

As a result, it was found that there is no difference in spontaneous alternation of Y-maze test. However, according to the result of ORT and OLT as long-term memory test, more enhanced cognitive ability was obtained compared to the normal mouse (FIGS. 11A to 11D).

### (6) Determination of the Effect of Glutamine Diet on Ameliorating Mild Cognitive Impairment

After the oral administration of glutamine following an occurrence of cognitive impairment that has been caused by chronic immobilization stress (CIS), the effect of treating cognitive impairment was examined.

Two weeks after applying CIS, the cognitive ability was tested by ORT-OLT. After that, while orally administering glutamine for 1 week (45 mg glutamine/kg bodyweight/day), a change in cognitive ability was examined from Day 4 (i.e., ORT-OLT were carried out from Day 4 to Day 7).

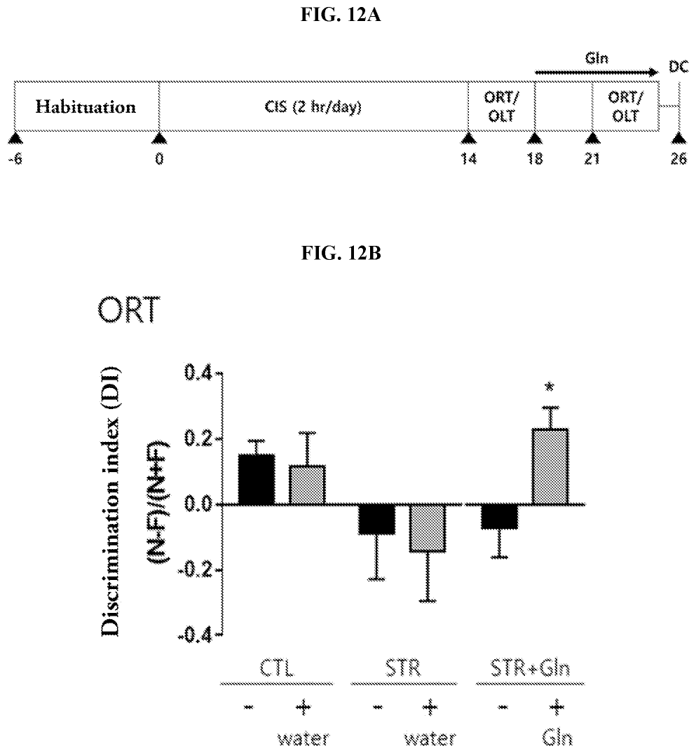

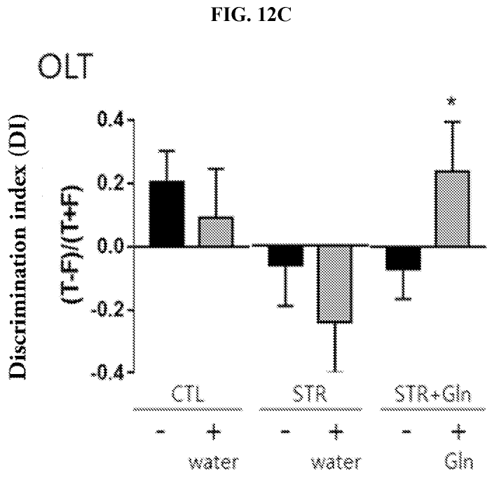

The chronic stress group applied with stress for 2 weeks was found to have a decrease in cognitive ability according to both the ORT and OLT. On the other hand, the group orally administered with glutamine for 1 week thereafter showed the cognitive ability restored to the level of stress-free control group. Based on this result, it was confirmed that the glutamine diet is effective for prevention and treatment of mild cognitive impairment (FIGS. 12A to 12C).

1-8. (canceled)

9. A method for ameliorating brain damage and mild cognitive impairment, the method comprising:
administering a composition comprising glutamine or a salt thereof to a subject in need thereof.

10. The method of claim 9, wherein the brain damage and mild cognitive impairment are caused by stress.

11. The method of claim 9, wherein the brain damage and mild cognitive impairment are caused by accumulation of amyloid beta 42 (Aβ42).

12. The method of claim 9, wherein the mild cognitive impairment is a decline in one or more function selected from memory, cognitive ability, and learning ability.

13. The method of claim 9, wherein the composition is in any formulation selected from the group consisting of a pill, a tablet, a capsule, a powder reparation, powder, a granule, a candy, a syrup, and a drink.

14. The method of claim 9, wherein the composition is a pharmaceutical composition comprising the glutamine or a pharmaceutically acceptable salt thereof.

15. The method of claim 14, wherein the composition further comprises, in addition to the effective component, a pharmaceutically acceptable carrier, a pharmaceutically acceptable vehicle, or a pharmaceutically acceptable diluent.

16. The method of claim 9, wherein the composition is a quasi-drug.

17. The method of claim 9, wherein the composition is included in a functional health food supplement.
